(19) United States
(12) Patent Application Publication
(10) Pub. No.: US 2002/0072758 A1
(43) Pub. Date: Jun. 13, 2002

(54) Processes for producing anastomotic components having magnetic properties

(76) Inventors: Michael L. Reo, Redwood City, CA (US); Dean F. Carson, Mountain View, CA (US); David H. Cole, San Mateo, CA (US); A. Adam Sharkawy, Union City, CA (US); Darin C. Gittings, Sunnyvale, CA (US)

Correspondence Address: HOEKENDIJK & LYNCH, LLP, P.O. Box 4787, Burlingame, CA, 94011-4878, US

(21) Appl. No.: 09/851,400

(22) Filed: May 7, 2001

Related U.S. Application Data
(60) Provisional application No. 60/255,635, filed on Dec. 13, 2000.

(51) Int. Cl. A61B 17/08

(52) U.S. Cl. 606/153

(57) ABSTRACT

Processes for forming magnetic anastomotic components subject suitable stock material to a series of steps to produce a biocompatible device for implantation in a patient's body. The processes treat the exterior of the material to form a smooth outer surface free of irregularities and discontinuities. The anastomotic component may undergo additional processing, for example, applying one or more coatings, changing the surface configuration of the component or creating one or more areas of concentrated magnetic flux. According to another aspect of the invention, the anastomotic component is magnetized after it has been packaged. Finally, the exterior of the component may be rendered smooth after initial forming by an acid etching process that is carried out pursuant to principles of the invention.

### CROSS-REFERENCE TO RELATED APPLICATIONS

This application claims priority under 35 USC § 120 from the following copending U.S. patent applications: “Methods and Devices Using Magnetic Force to Form an Anastomosis Between Hollow Bodies,” Ser. No. 09/562,599, filed Apr. 29, 2000 (Atty. Docket No. 015); “Magnetic Components for use in Forming Anastomoses, Creating Ports in Vessels and Closing Openings in Tissue,” Ser. No. 09/638,805, filed Aug. 12, 2000 (Atty. Docket No. 018); and “Anastomotic Components, Systems and Methods,” Serial No. 60/255,635, filed Dec. 13, 2000 (Atty. Docket No. 019P). The entire subject matter of each of these patent applications is expressly incorporated herein by reference.

### BACKGROUND OF THE INVENTION

1. Field of the Invention

The present invention relates generally to magnetic anastomotic components for use in making connections to or between various anatomical structures, and more particularly to processes for producing such components.

2. Description of Related Art

Attempts to manufacture a sutureless anastomotic coupler that provides the results and reliability of conventional handsewn anastomoses date back many years. Sutured anastomoses are the accepted manner for creating an anastomosis between hollow vessels, particularly in the field of vascular surgery. Some of the sutureless anastomotic systems developed more recently have reported encouraging initial results. To date, however, none of these connectors has performed well enough clinically to receive wide or even a significant level of acceptance in the field.

Among the many problems associated with known devices are acute or chronic occlusion of the coupler or adjacent vessel, as well as mechanical failure prior to, during or after delivery of the coupler or graft. Additionally, many of the proposed couplings, in addition to not remaining patent, penetrate or damage the target vessel wall, fail to produce a fluid-tight seal with the vessel, or are cumbersome and difficult to use.

In view of the performance challenges imposed by the environment in which such anastomotic couplers are used, there is a need for improved processes that facilitate and enable the production of couplers or components having adequate structural characteristics while providing the ability to effect rapid and secure connection of vessels.

### SUMMARY OF THE INVENTION

The invention provides processes for producing anastomotic components having magnetic properties that are used to couple hollow bodies quickly and securely to obtain reliable, fluid-tight connections. Anastomotic components manufactured pursuant to principles of the invention allow a practitioner to form an elegant yet clinically effective anastomosis with the flexibility to accommodate a wide range of vessel sizes and tissue types.

One embodiment of the invention provides a process for producing a magnetic anastomotic component for implantation in a patient's body and includes steps of forming an anastomotic component from a material capable of producing a magnetic field, processing the component to make its exterior surface suitable for receiving a layer of biocompatible material, and providing the exterior surface of the component with a layer of biocompatible material.

Another embodiment of the invention provides a process for producing a magnetic anastomotic component for implantation in a patient's body. This process includes steps of forming an anastomotic component having a desired configuration from a material capable of producing a magnetic field, packaging and sterilizing the component, and magnetizing the component in the package.

Still another embodiment of the invention provides a process for producing a magnetic anastomotic component for implantation in a patient's body. This process has steps of providing an anastomotic component having an ability to produce a magnetic field and an exterior surface, placing a layer of material on a first portion of the exterior surface of the component so as to leave a second portion of the exterior surface of the component uncovered by the material, and magnetizing the component.

Yet another embodiment of the invention provides a process for producing a magnetic anastomotic component that includes steps of forming an anastomotic component from a material capable of producing a magnetic field, subjecting the exterior surface of the component to an acid etching process to remove surface irregularities, and providing the exterior surface of the component with a layer of biocompatible material.

### BRIEF DESCRIPTION OF THE DRAWINGS

These and other aspects, benefits, advantages and features of the invention will be apparent from the following description of drawing figures taken in conjunction with the accompanying detailed description of preferred embodiments, wherein:

FIG. 1 is a flow chart illustrating a series of process steps carried out according to one embodiment of the invention;

FIG. 2 is a flow chart illustrating a series of process steps carried out according to another embodiment of the invention;

FIG. 3 is a flow chart illustrating a series of process steps carried out according to yet another embodiment of the invention;

FIGS. 4A-4E are perspective views of exemplary anastomotic components that may be produced according to one or more embodiments of the invention;

FIGS. 5A-5C are elevation views in section showing a portion of material having magnetic properties being processed according to an embodiment of the present invention;

FIGS. 6A-6B are perspective views showing, respectively, an anastomotic component before and after the exterior surface has been prepared for subsequent processing according to one embodiment of the present invention;

FIGS. 7A-7B are perspective views showing, respectively, the anastomotic component shown in FIG. 6A before and after it has been prepared for subsequent processing according to another embodiment of the present invention;

FIGS. 8A-8B are elevation views in section showing a particular processing step being performed on a component according to an embodiment of the invention;

FIGS. 9A-9B are, respectively, elevation views in section showing one and two bar magnets along with lines representing the magnetic field;

FIGS. 10A-10B are, respectively, elevation views in section of the bar magnets shown in FIGS. 9A-9B coated with a material having a relatively high magnetic permeability;

FIGS. 11A-11C are elevation views in section sequentially illustrating processing a magnetic material according to one embodiment of the invention to obtain one or more areas of concentrated magnetic flux;

FIGS. 12-14 are, respectively, elevation views in section depicting magnet pairs as shown in FIGS. 9B, 10B and 11C positioned on opposite surfaces of a wall of tissue;

FIG. 15A is a perspective view of an anastomotic component constructed according to another embodiment of the invention;

FIG. 15B is a sectional view of the anastomotic component shown in FIG. 15A mounted on a hollow vessel;

FIG. 16A is a perspective view of an anastomotic component constructed according to still another embodiment of the invention; and

FIG. 16B is a sectional view of the anastomotic component shown in FIG. 16A mounted on a hollow vessel.

### DETAILED DESCRIPTION OF THE PREFERRED EMBODIMENTS

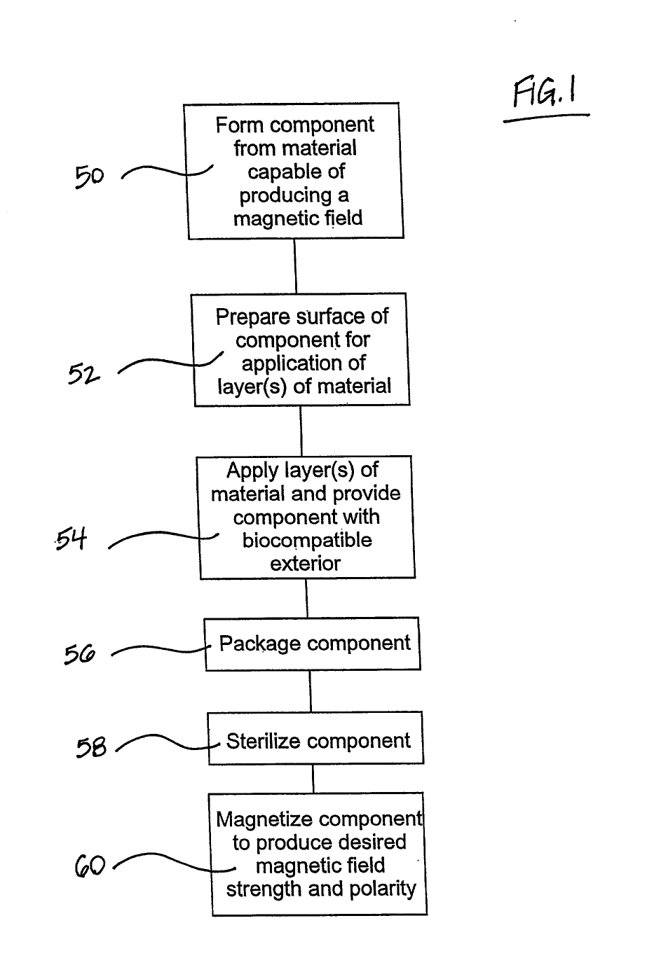

FIGS. 1-3 are flow charts that schematically illustrate various series of process steps carried out according to different embodiments of the invention, the processes producing an anastomotic component having magnetic properties, i.e., the component has the ability to produce or be affected by a magnetic field.

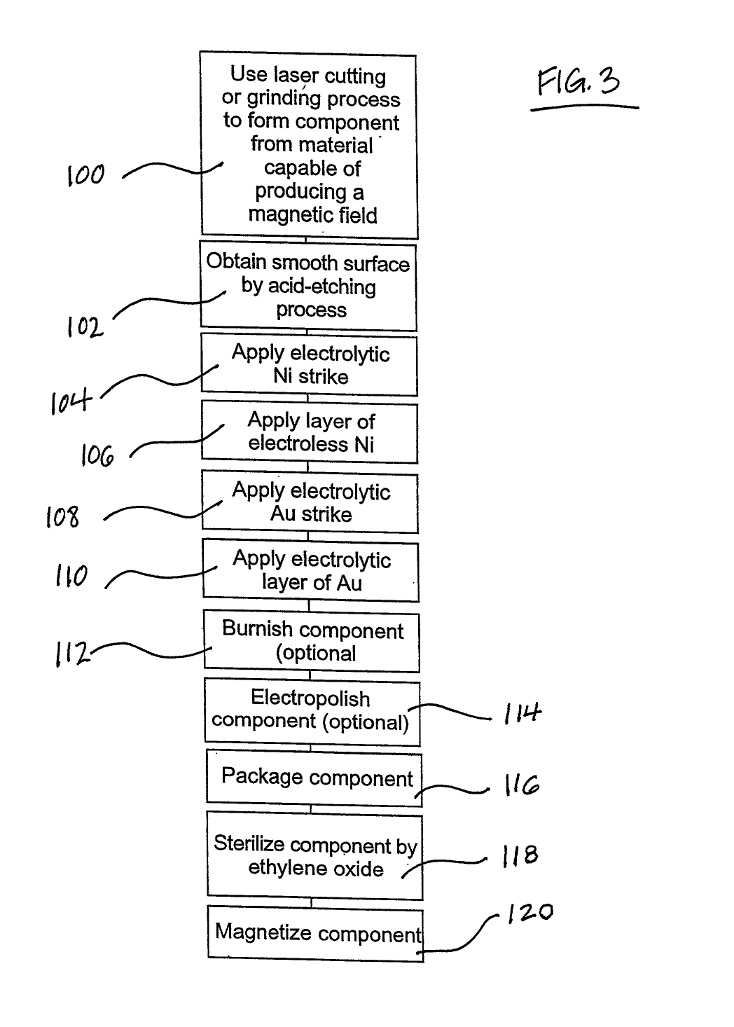

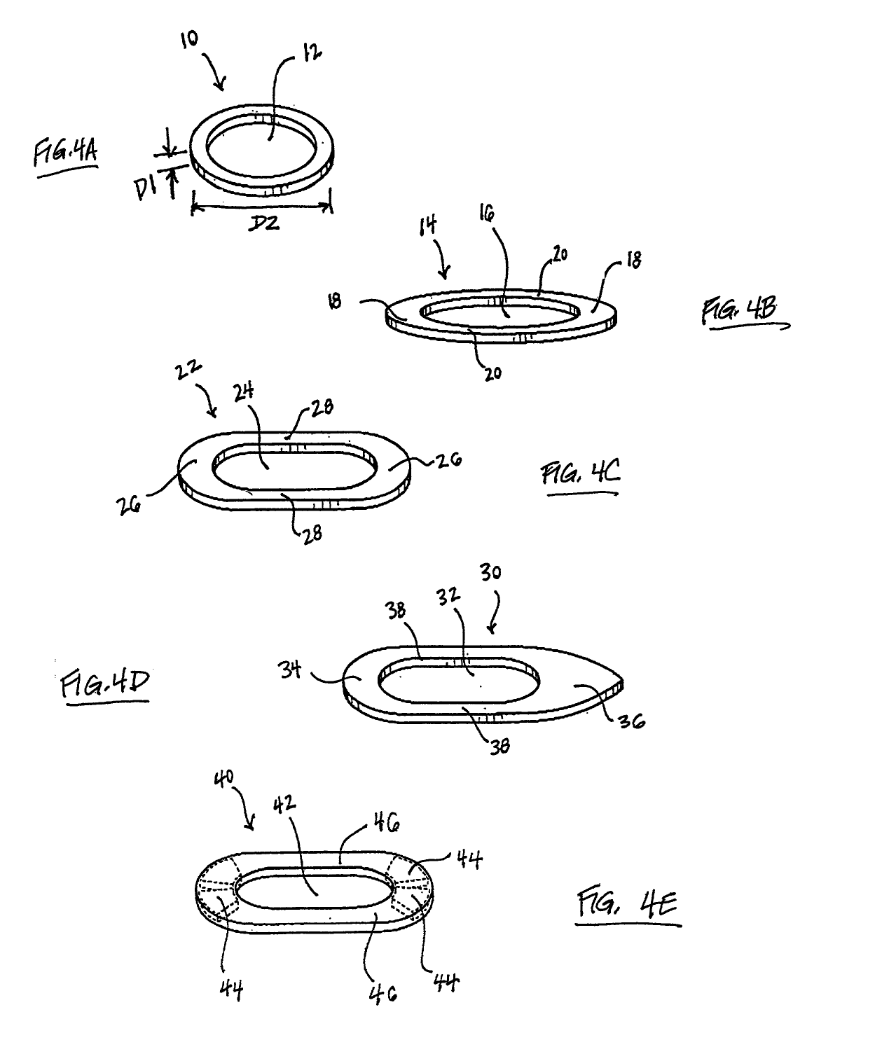

Before describing the flow charts of FIGS. 1-3, though, attention is turned to FIGS. 4A-4E and the anastomotic components shown therein. A brief discussion of the illustrated anastomotic components follows. For a more detailed description of the components see aforementioned co-pending application Ser. No. 09/562,599 (incorporated by reference above).

As discussed therein, suitable materials that may be used to form the anastomotic component include NdFeB (Neodymium Iron Boron), SmCo (Samarium Cobalt), and Alnico (Aluminum Nickel Cobalt), with NdFeB being more preferred than the other two materials. It should be understood that various parameters, for example, the particular material used, the size, configuration and processing of the component, etc., will be selected and varied based on the desired characteristics of the finished component; e.g., the magnitude, distribution, location, etc., of the magnetic force.

FIGS. 4A-4E illustrate several exemplary embodiments of anastomotic securing components for use in forming an anastomosis between first and second hollow bodies. FIG. 4A shows a component 10 with an annular body and an opening 12 defined by the body. The component 10 is generally ring-shaped and is circular in plan view with a constant (or substantially constant) thickness around its perimeter. The size of the opening 12 is preferably large to allow ample flow without comprising magnetic force beyond an acceptable level. The component 10 is sized and configured to be placed adjacent an opening of a first hollow body that has been prepared for anastomosis to a second hollow body. In the preferred embodiment, a second component (not shown) would be placed adjacent an opening of the second hollow body for making the anastomotic connection.

FIG. 4B shows an elliptical-shaped, anastomotic component 14 with an opening 16. The component 14 is generally plate-shaped and the opening 16 is configured to provide the component 14 with larger end portions 18 than side portions 20. FIG. 4C shows an oval or racetrack-shaped component 22 with an opening 24. As in component 14, the opening 24 provides component 22 with larger end portions 26 than side portions 28. The end portions of either embodiment may have the same width as the remaining portion of the component to increase the size of the opening.

FIG. 4D shows an anastomotic component 30 with an opening 32, two end portions 34, 36 and two side portions 38. The securing component 30 has a generally racetrack-shaped configuration; however, the end portion 36 is larger than the end portion 34, giving the component 30 an asymmetric configuration. That is, the opening 32 is not centrally located with respect to the body of the component 30, unlike the openings 12, 16 and 24 of respective securing components 10, 14 and 22 (shown in FIGS. 4A-4C). Also, the end 36 of component 30 provides a tapered leading edge for easier introduction into a hollow body such as blood vessel.

It will be understood that according to the processing aspects of the present invention, the specific composition, shape and size of the anastomotic components, in whole or in part, may be varied in many fashions from the exemplary configurations depicted in FIGS. 4A-4D. For example, the thickness or width of the component may stay constant or change along all or part of the body of the component. The anastomotic components of the invention are preferably, though not necessarily, plate-shaped, i.e., their thickness is less than their length, width, or diameter. A diameter Dl corresponding to the thickness of the component in FIG. 4A is less than a dimension D2 corresponding to the diameter of the component. While it is desirable in most applications to minimize the thickness of the component, the invention may be practiced to produce an anastomotic component having a relatively large thickness.

It will be noted that the anastomotic components shown in FIGS. 4A-4D are generally flat; however, they could instead be curved or arcuate, or comprise a combination of flat and curved sections. Additionally, the shape of each component may be different from the shape of the opening therein, unlike the embodiments of FIGS. 4A-4D. Finally, anastomotic components produced according to the invention may include more than one opening if desired. In sum, myriad potential considerations may impact the specific implementation or use of the inventive processes described herein to produce a particular anastomotic component.

The preferred embodiments of the invention are described with respect to a material having magnetic properties. It should be understood that this is for clarity and explanatory purposes only, as the component produced according to the invention may be formed of, have incorporated therein (or otherwise be combined with) a material capable of producing a magnetic field. As an example, each of the anastomotic components shown in FIGS. 4A-4C is formed substantially entirely of a magnetic material such that magnetic force may be generated over the entire area of the component. FIG. 4E shows an alternative embodiment wherein a component 40 with an opening 42 has several magnetic members 44 located at discrete areas of the component. The remaining areas 46 of the component 40 may thus be formed of a different material. This aspect of the invention may be useful in producing a flexible or collapsible anastomotic component, for example, by making areas 46 of component 40 flexible.

Returning to the flow chart of FIG. 1, step 50 represents forming an anastomotic component from suitable stock material, as discussed above. The anastomotic component is then subjected to a process that prepares its exterior surface to receive a layer of biocompatible material that renders the component safe for implantation in a patient. The step 52 represents this preparatory process, which in preferred embodiments may be characterized as treating the anastomotic component to remove unwanted surface irregularities and debris.

One option for cleaning the anastomotic component is mechanical finishing to remove the material from the surface of the component. A mechanical finishing process, though, carries with it the inherent risk of removing too much of the component along with the unwanted material. For instance, it is not uncommon for square comers to become rounded as a result of mechanical finishing. A flat surface and sharp corners are desirable to maximize magnetic force and sealing between a component and tissue (or another component). It is, however, to round the comers to some extend for atraumatic deployment. Accordingly, the present invention provides processes using either or both mechanical and non-mechanical finishing on the component.

With reference again to FIG. 1, subsequent to preparing the surface of the anastomotic component at 52, a layer of biocompatible material is applied to the exterior of the component at 54. The anastomotic component is then packaged at 56, sterilized at 58 and magnetized at 60, while packaged, to give the component the desired magnetic strength and polarity. It should be recognized that FIG. 1 represents the invention broadly with some preferred aspects, and is intended to provide context for the more detailed discussions below of individual process steps that may be carried out according to specific embodiments.

FIG. 2 is a flow chart depicting a more specific example of a process performed according to some embodiments of the invention. The forming step 70 comprises subjecting NdFeB stock material to an EDM process to form an anastomotic component having a desired configuration, size, etc. It should be noted that the anastomotic component may also be formed into a configuration that is designed to be further processed in order to produce the final component. As an example, an anastomotic component of NdFeB may be formed with a relatively flat surface that is intended to contact tissue. A subsequent processing step (for instance, acid-etching) may be used to modify this surface of the anastomotic component to produce traction elements adapted to grasp tissue and enhance engagement between the component and the vessel (for example, as shown in FIGS. 16A-16B).

The anastomotic component formed by the process of FIG. 2 is next subjected to a micro-abrasive process 72 that provides the component with a substantially smooth exterior surface. The particular type of micro-abrasive (or other mechanical finishing) procedure used may vary from those discussed herein, as those skilled in the art will appreciate. The anastomotic component is rendered biocompatible at 74 by applying a suitable coating or layer, is packaged at 76, magnetized at 78 and sterilized at 80 by radiation, e.g., by gamma or electron beam. The exemplary process of FIG. 2 is described additionally below in connection with Example 1.

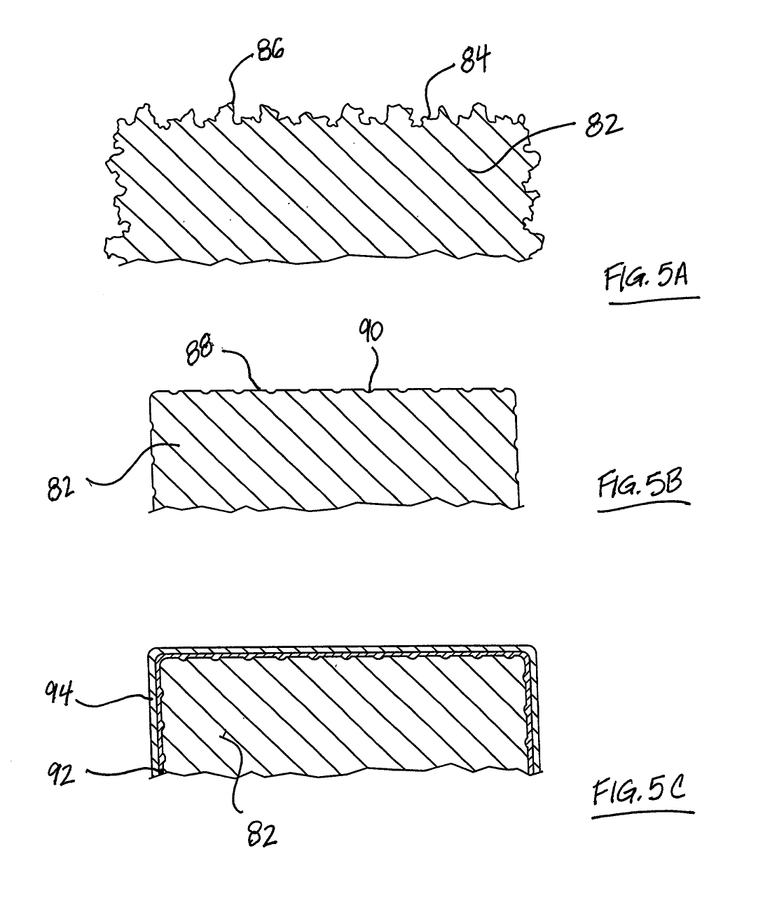

According to this embodiment of the invention, also illustrated schematically in FIG. 5A, an anastomotic component is cut from NdFeB stock using an EDM process. As the cutting wire (not shown) used by the EDM process cuts through the material to form the component 82, the wire melts to some extent, leaving unwanted material on the surface of the component. As shown in FIG. 5A, the melted material of the wire combines with the NdFeB to create a mixture 84 that firmly adheres to (or becomes mixed with) the exterior surface of the component. This mixture 84, which may be referred to as “recast” and typically has an irregular surface 86 with jagged edges (exaggerated for clarity in FIG. 5A), is removed prior to providing the component with a biocompatible layer. This prevents the added layer is from simply conforming to the irregular surface beneath it.

As noted above, one preferred embodiment of the invention removes the unwanted material 84 and smoothes the surface 86 of the anastomotic component 82 by subjecting it to a mechanical finishing process which places the formed components in a mechanical abrasive environment. This environment, for example, a tumbler, subjects the exterior surface of the anastomotic component to repeated and violent collisions with selected media (e.g., steel or glass spheres) moving at an extremely high velocity. FIG. 5B shows the component 82 after the micro-abrasive procedure discussed above has produced a much smoother surface 88 than existed after initially forming the component.

FIG. 5B shows, however, that the surface 88 of the component 82 still contains irregularities to a certain degree given the presence of pits 90 that form depressions (again, exaggerated in the Figures for clarity). The next step 54 in FIG. 2 provides the component 82 with one or more layers 92, 94 of desired material, the layers being applied in any suitable manner. The material(s) forming layers 92, 94 provide the anastomotic component with an exterior surface that is biocompatible and, preferably, very smooth and substantially free of imperfections or irregularities, as schematically represented by the surface component 82 of FIG. 5C.

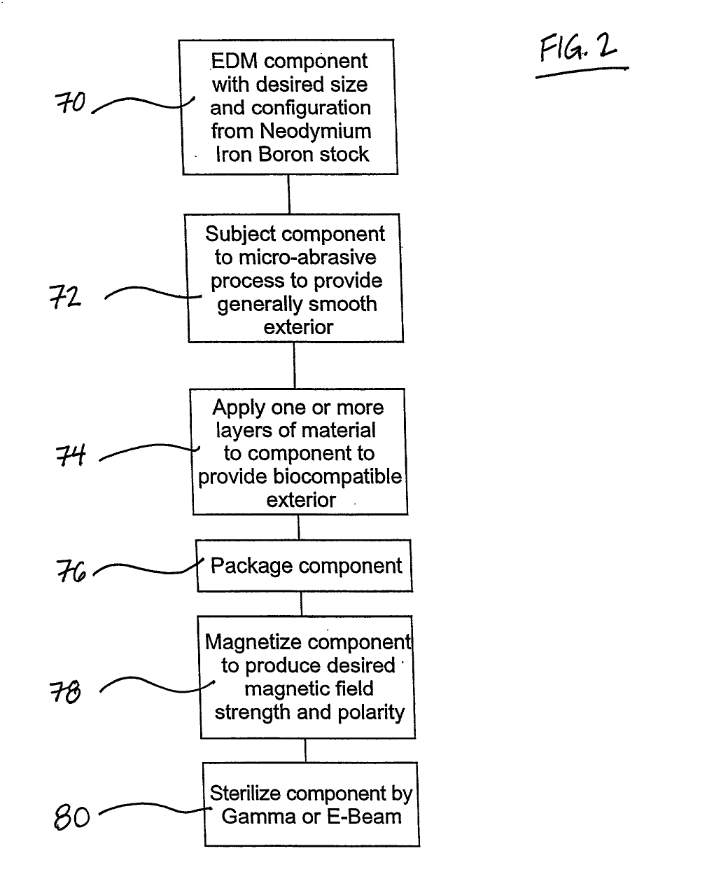

FIG. 3 is a flow chart depicting a specific example of a process performed according to another embodiment of the invention. The forming step 100 comprises laser cutting or grinding suitable stock magnetic material to form the anastomotic component. The component receives various layers of material at steps 104, 106, 108 and 110, is burnished or peened at 112, electropolished at 114, packaged at 116, sterilized by ethylene oxide (or other gas) at 118 and magnetized at 120. This exemplary process is described further below in connection with Example 2. The finished anastomotic component may then be packaged, for instance, with other similarly (or differently) configured anastomotic components, a delivery device, etc., as part of a kit used for one or more given procedures.

As is the case with forming the anastomotic component of the invention using an EDM process, laser cutting and grinding may also produce an anastomotic component having an exterior surface containing irregularities that render the component unsuitable for use. According to this embodiment, rather than (or in addition to) micro-blasting or using an abrasive procedure to smooth the exterior surface, an acid etching process 102 is used to obtain the desired surface configuration on the component.

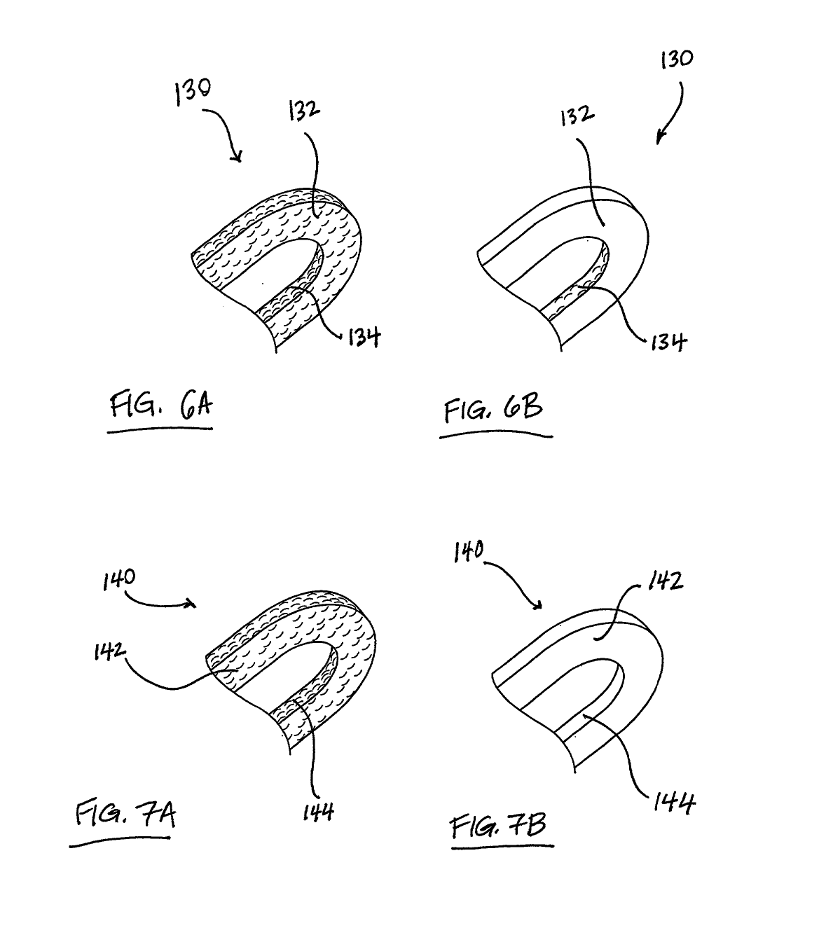

FIG. 6A shows an anastomotic component 130 after it has been formed, for example, by EDM, laser cutting, grinding, etc. The anastomotic component 130 has an exterior surface 132 including an area 134 around the inner diameter of the component. As can be seen from FIG. 6A, virtually the entire exterior surface of the anastomotic component 130 has irregularities of the type previously discussed. FIG. 6B shows the component 130 after it has been subjected to a mechanical finishing process that failed to remove all surface irregularities at the area 134.

The reason for inadequate cleaning or smoothing at the area 134 is that the media used to mechanically finish the component surface do not fully access and thus treat this area. As an example, if the anastomotic component is formed and then placed in a tumbler containing highly abrasive media, such as steel or glass spheres or beads, there is a tendency for the media not to reach all the surfaces around the inner diameter or opening in the component. Consequently, surface irregularities at least partially remain at this area. Further, these mechanical finishing processes usually remove material at the perimeter first and then at the center portion of the anastomotic component. In some cases this can lead to removing too much material at the edges or corners of the component, which may compromise magnetic force, eliminate desired structural features, adversely affect sealing capabilities, etc.

FIG. 7A shows an anastomotic component 140 with an exterior surface 142 and an area 144 that corresponds to the area 134 of the component 130, the entire exterior essentially being covered with unwanted material. FIG. 7B shows the component 140 after it has been subjected to an acid etching procedure. As can be seen the result is that the entire exterior surface 142 is substantially free of irregularities, including the area 144 that some mechanical finishing processes may fail to fully treat. Any suitable acid bath may be used to practice this aspect of the invention, with phosphoric acid being preferred. A specific example of an acid-etching process according to one embodiment of the invention is described below with respect to Example 2.

While the embodiments represented by FIGS. 2 and 3 use one type of surface treatment, either micro-abrasion or acid etching, it will be understood that according to the invention these processes may be used apart or together. As an example, the invention may be practiced by first subjecting the component to acid-etching, and then a relatively light mechanical finishing process to minimize excessive material removal. Similarly, other aspects of the invention that are described in connection with the illustrated embodiments may be used independently or in conjunction with one another, either as shown and discussed herein or otherwise.

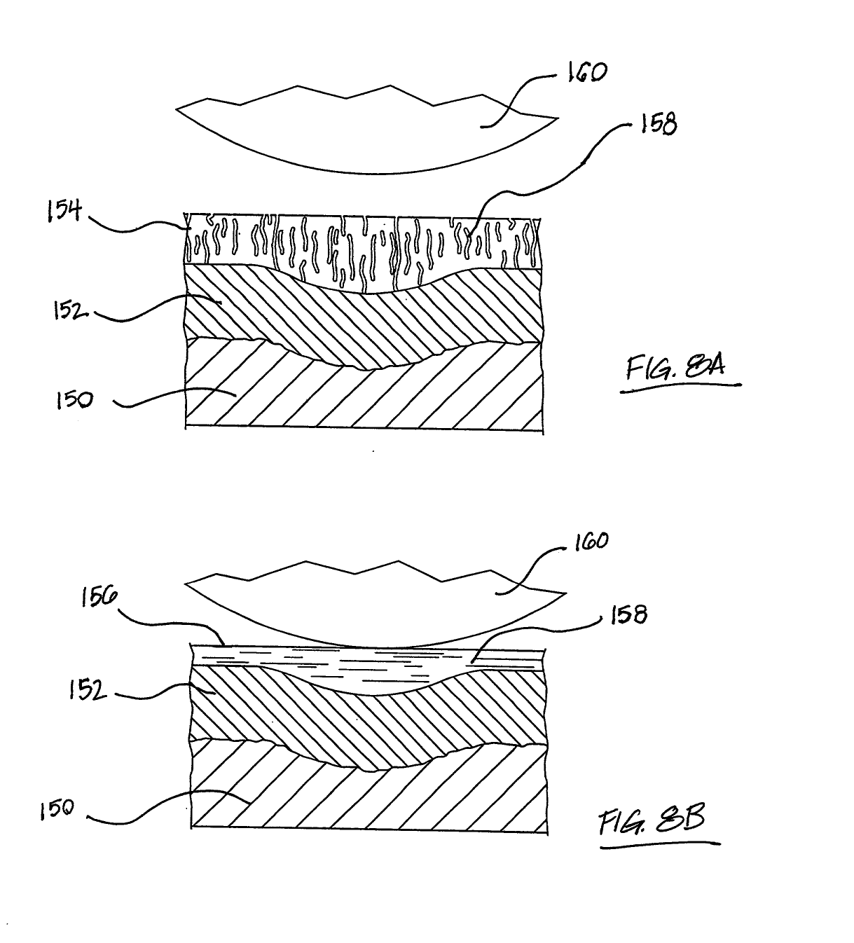

FIGS. 8A-8B show an anastomotic component 150 comprising a base magnetic material with a first layer 152 and a second layer 154, the layers 152, 154 being different materials. The exterior surface 156 defined by the layer 154 is smooth and generally free of irregularities, save for microscopic pores or interstices 158 which may be present in one or more of the layers on the component. For sake of example, if first layer 152 is Nickel (not biocompatible) and layer 154 is Gold (biocompatible), these pores 158 are undesirable because the Nickel could bleed through the Gold resulting in possible adverse effects to the patient.

Accordingly, another embodiment of the invention, illustrated in FIG. 8B, overcomes this concern by subjecting the anastomotic component 150 to a burnishing or peening operation to crush the Gold (very ductile), thereby eliminating the pores and reducing the chance of Nickel bleeding out. The ball 160 represents a surface that is peened or hammered against the exterior surface 156 of the anastomotic component 150, smashing the layer of Gold 154 and crushing the pores as stated above.

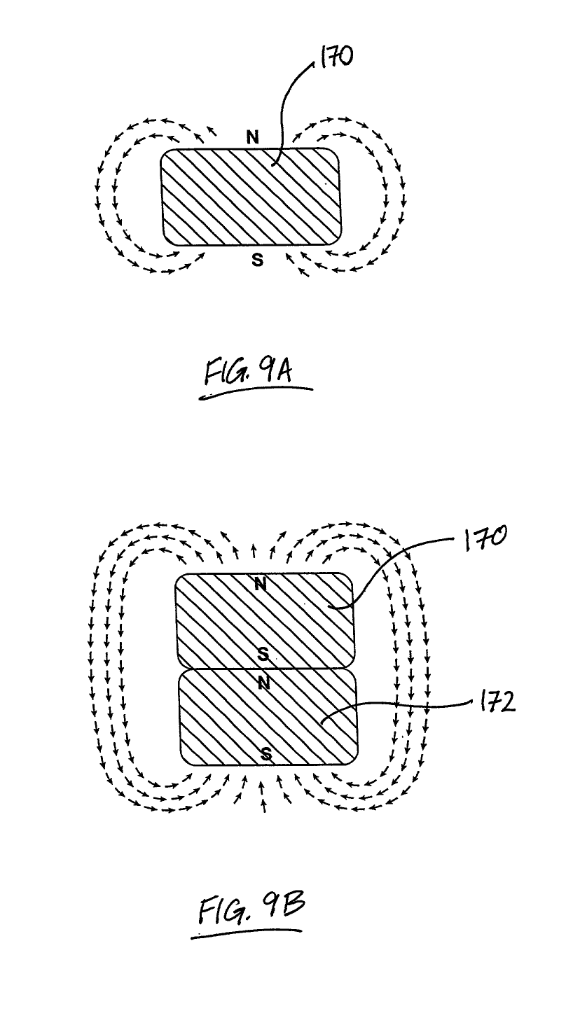

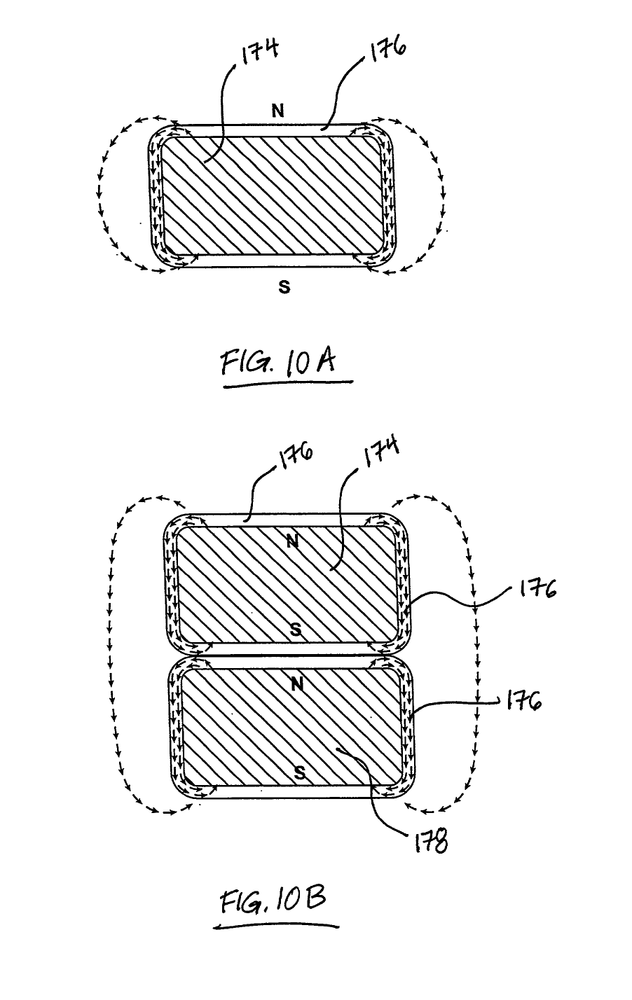

FIG. 9A shows a bar magnet 170 with its poles labeled N and S and the magnetic field represented by broken lines 172. FIG. 9B shows the magnet 170 attracted to a second magnet 172 along with the magnet field produced by the combined fields of the magnets. These magnets (and those of FIGS. 10A-lOB) are shown to explain another aspect of the invention.

FIG. 10A shows a bar magnet 174 the exterior of which is provided with a layer of material 176. The material 176, e.g., Nickel, has a high magnetic permeability such that the magnetic flux is shunted into it. That is, comparing FIGS. 9A and 10A, it can be seen that the layer of Nickel (or other material having a high permeability) significantly decreases the strength of the magnetic field. FIG. 10B shows plainly the effect such a layer or coating has when placed on each of a pair of magnets 176, 178; the coating shunts the magnetic field and reduces the magnetic flux that attracts the magnets.

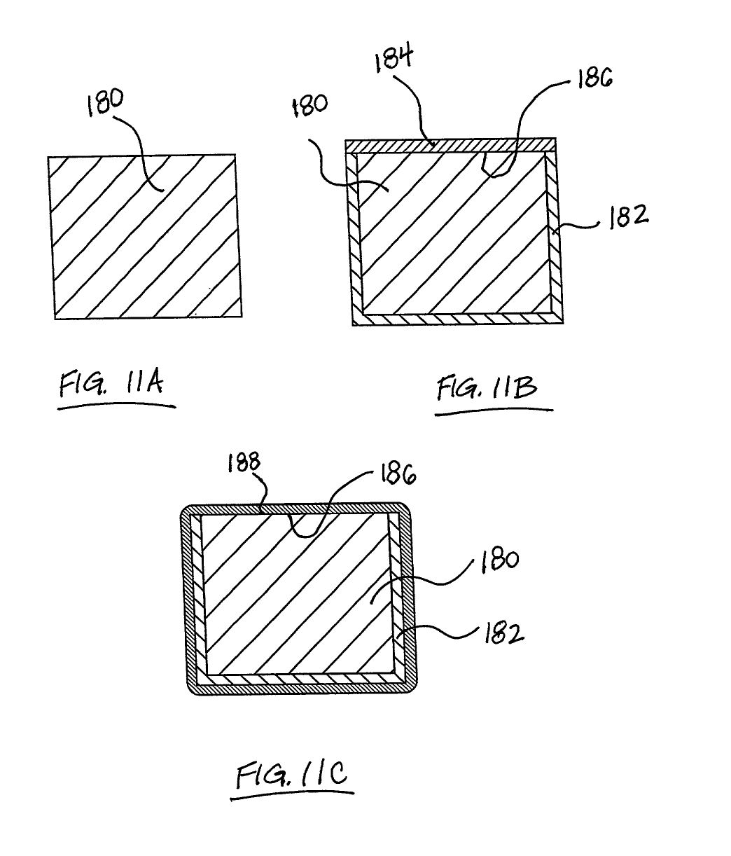

FIGS. 11A-11C show schematically a process for producing an anastomotic component that produces a magnetic field that is concentrated in a particular area of the component. A first portion of the component is provided with a layer of material having a high magnetic permeability, while a second portion is not covered with the material. FIG. 11A shows base material 180 (representative of an anastomotic component) and FIG. 11B shows the material 180 after a layer of material 182 has been placed over a first portion of its exterior. A masking layer 184 is placed over a second portion 186 of the material 180 so that the material 182 is not applied to the second portion. FIG. 11C shows the base material 180 after the masking layer 184 has been removed and an additional layer 188 has been applied over the entire exterior surface. As can be seen, the second portion 186 has a different construction than the remaining portion of the component.

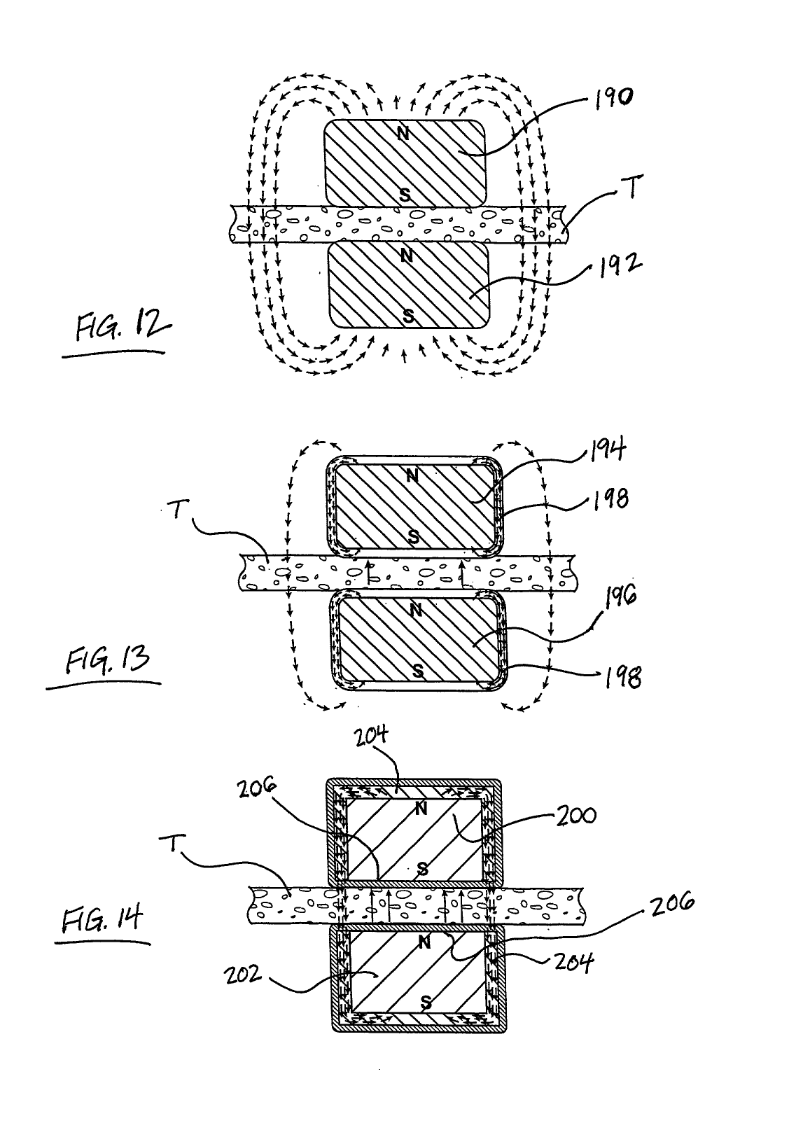

FIGS. 12-14 show an exemplary application of the embodiment shown in FIG. 11C. FIG. 12 is a view showing two plain bar magnets 190, 190 sandwiching a wall of tissue T. The magnetic flux between the magnets 190, 192 is illustrated by the broken lines and is indicative of the attraction force between the magnets. FIG. 13 shows two magnets 194, 196 each of which has a layer 198 of a material having a relatively high magnetic permeability. As shown, the presence of this material drastically decreases the magnetic attraction force between the magnets 194, 196.

FIG. 14 shows two magnets 200, 202 placed on opposite sides of the tissue wall T. Each magnet 200, 202 has a layer 204 of material having a high permeability, e.g., Nickel. The magnets however have a portion 206 that is not covered by the layer 204, pursuant to FIGS. 11A-11C. A layer of biocompatible material, e.g., Gold, overlies the entire exterior. As shown by FIG. 11C, the magnetic flux concentrates at the portions 206 of the magnets 200, 202, and more particularly, at the ends thereof (to the right and left as viewed in FIG. 11C). As Gold is a diamagnetic, the magnetic field lines pass through the Gold at portions 206. This flux concentration increases the force of attraction and allows use of a smaller overall component, as discussed in aforementioned co-pending application Ser. No. 09/638,805 (the contents of which have been incorporated by reference).

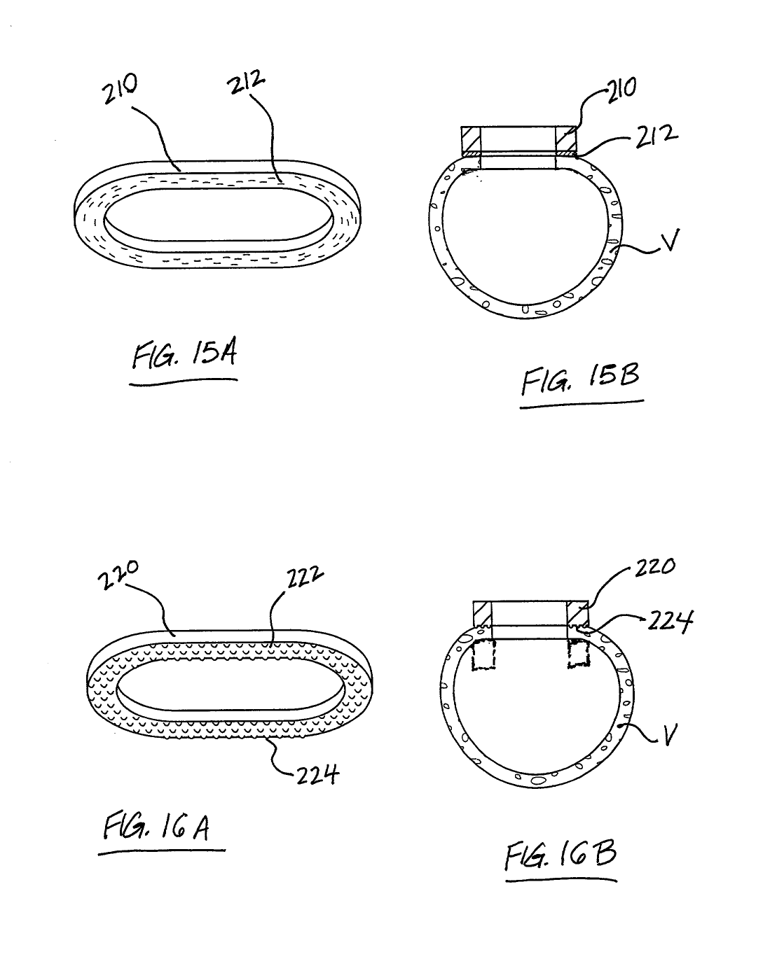

FIGS. 15A-15B show an anastomotic component 210 produced by a process performed according to another embodiment of the invention. The component 210 has a surface adapted to be adhered to a vessel in order to secure the component. The surface is provided with a layer of bioadhesive 212 that fixes the component 210 to a vessel V, as shown in FIG. 15B. Embodiments relating to placing an adhesive or other extravascular magnetic anastomotic component are disclosed in detail in aforementioned co-pending application Serial No. 60/255,635 (the contents of which have been incorporated by reference).

FIGS. 16A-16B show an anastomotic component 220 produced by a process performed according to still another embodiment of the invention. The component 220 has a surface 222 adapted to contact the wall of a vessel once the component is secured thereto. (It will be noted that the component 220 may be secured to the vessel wall by any suitable means, for example, via the magnet field of an adjacent magnet, such as the intraluminal magnet shown in phantom in FIG. 16B.) The surface 222 is provided with some form of mechanism or structure for enhancing engagement between the component 220 and the vessel. In FIG. 16A the mechanism comprises small projections 224 which, as shown in FIG. 16B, grip the tissue of the vessel. It will be recognized that various manners of enhancing the engagement may be used instead of or in addition to the projections 224. For example, the bioadhesive 212 that acts to secure the component 210 to the vessel wall, or a layer of tacky material, could be used in conjunction with other securing means. Other enhance mechanisms or structures include roughened or knurled surfaces, barbs, hooks, pins, etc.

### EXAMPLE 1

An example of a process carried out according to one embodiment of the invention will now be described. This example generally corresponds to the process represented by FIG. 2. A tube of desired geometry is cut via wire EDM from a block of raw material, e.g., NeoFeB. The tube is between one and two inches long, and, when viewed endon, has a profile matching that of the magnets cut from it. The tube is sectioned via wire EDM into individual magnets having a desired thickness, e.g., 0.015±0.005 inches. These parts are cut slightly oversize to allow for material removal in subsequent operations. The individual components are then placed in a centrifugal finishing machine with a fine grit silicone carbide abrasive media. The machine is run at a high speed producing a uniform mechanical abrasion to all of the surfaces of the parts. The amount of material removal is controlled by a variety of factors including processing time, speed of the equipment and media selection. After the surface of the component has bee prepared in the aforementioned fashion an electrolytic nickel strike is applied thereto. Typically the thickness of the nickel strike is 0.00003 to 0.00006 inches. Electroless nickel is applied directly to the nickel strike with a thickness of 0.0001 to 0.0006 inches. Next, a gold strike is applied to the electroless nickel layer. An exemplary thickness for the gold strike is 0.000004 to 0.000006 inches. Gold is then electrolytically applied to the gold strike to a thickness of approximately 0.0005 inches. The component is then placed in packaging that forms a sterile barrier around the component. The packaged component is then subjected to a magnetic field capable of magnetizing the component. Fields of 35-kilo oersteads are typical for magnetizing NeoFeB magnets. The packaged magnetized component is then subjected to gamma radiation or an electron beam to ensure the sterility of the component.

### EXAMPLE 2

An example of a process carried out according to another embodiment of the invention will now be described. This example generally corresponds to the process represented by FIG. 3. First, sheets of material are ground that are one by two inches and have a thickness between 0.010 and 0.020 inches. An Nd Yag laser is then employed to ablate material in a pattern that will produce the desired plan view profile of the part. It can be seen that multiple parts could be cut from a 1 by 2 sheet. These parts are cut oversize to allow for material removal in subsequent operations. The individual components are then placed in phosphoric acid for between 5 and 10 minutes to remove the surface layer of remolten or otherwise compromised material present. Controlling the amount of material removal is accomplished by controlling such process parameters as etching time, temperature of the acid and amount of agitation imparted to the acid. After the surface of the component has bee prepared in this fashion, an electrolytic nickel strike is applied to the surface. Typically the thickness of the nickel strike is 0.00003 to 0.00006 inches. Electro-less nickel is applied directly to the nickel strike, this layer having a thickness between 0.0001 and 0.0006 inches. A gold strike is applied to the electro less nickel layer, its thickness being between 0.000004 and 0.000006 inches. Gold is then electrolytically applied to the gold strike to a thickness of approximately 0.0005 inches. The component can then be burnished by subjecting the surface to being impacted by steel shot of a small size moving at a high velocity. This process differs from centrifugal finishing in that its goal, rather than remove material, is to only to plastically deform and compact it. If desired the component can be subjected to electro-polishing to further smoothe the surface. The component is then placed in packaging that forms a sterile barrier around the component. The packaging material is be of a special type that is permeable to ethylene oxide gas. The packaged component is then sterilized by exposing the package to ethylene oxide gas for a period of time. The packaged and sterilized component is then subjected to field capable of magnetizing the component. Fields of 35-kilo oersteads are typical for magnetizing NdFeB magnets.

The preferred embodiments of the invention are described above in detail for the purpose of setting forth a complete disclosure and for sake of explanation and clarity. It will be readily understood that the scope of the invention defined by the appended claims will encompass numerous changes and modifications.

What is claimed is:

1. A process for producing a magnetic anastomotic component suitable for implantation in a patient's body, the process comprising steps of:
forming an anastomotic component having a desired configuration from a material capable of producing a magnetic field, the anastomotic component having an exterior surface;
processing the anastomotic component to make the exterior surface suitable for receiving a layer of biocompatible material; and
providing the exterior surface of the anastomotic component with a layer of biocompatible material.

2. The process of claim 1 wherein the processing step is performed to make the exterior surface of the component substantially smooth.

3. The process of claim 2 wherein the processing step comprises removing unwanted material from the exterior surface of the component by abrasive microblasting.

4. The process of claim 3 wherein the processing step comprises placing the component in a mechanically abrasive environment.

5. The process of claim 2 wherein the processing step comprises grinding the exterior surface of the component.

6. The process of claim 2 wherein the processing step comprises acid etching the exterior surface of the component.

7. The process of claim 1 wherein the providing step comprises disposing a layer of biocompatible material over another layer of material that covers the exterior surface of the anastomotic component.

8. The process of claim 7 wherein the layer of biocompatible material is Gold and the other layer of material is Gold or Nickel.

9. The process of claim 1, further comprising electropolishing the component after placing a final layer of material thereon.

10. The process of claim 1 wherein the component has an overall thickness within the range of from about 0.010 to about 0.030 inch, and the biocompatible layer has a thickness within the range of from about 0.0002 to about 0.0020 inch.

11. The process of claim 1 wherein the component is formed from NeoFeB and a layer of biocompatible material is placed over the NeoFeB.

12. The process of claim 1 wherein a portion of the exterior surface is formed with means for enhancing engagement between the component and the tissue of a vessel.

13. The process of claim 1 wherein the forming step forms a component comprised entirely of a material capable of producing a magnetic field.

14. The process of claim 1 wherein the forming step forms a component having a first configuration and the processing step changes the component to a second configuration having structural differences from the first configuration.

15. The process of claim 1 wherein the providing step comprises plating the exterior surface of the component.

16. The process of claim 15 wherein the exterior surface of the component is plated more than once.

17. The process of claim 1 wherein further comprising assembling the anastomotic component is assembled with a delivery device for packaging and sterilization.

18. The process of claim 1 wherein the anastomotic component is packaged and sterilized after the providing step.

19. The process of claim 18 wherein the component is magnetized either before or after being packaged and sterilized.

20. A process for producing a magnetic anastomotic component suitable for implantation in a patient's body, the process comprising steps of:
forming an anastomotic component having a desired configuration from a material capable of producing a magnetic field;
packaging the component;
sterilizing the component; and
magnetizing the component in the package.

21. The process of claim 20 wherein the anastomotic component is packaged, magnetized and then sterilized.

22. The process of claim 21 wherein the component is packaged, sterilized and then magnetized.

23. The process of claim 22 wherein the component is sterilized by gas.

24. The process of claim 21 wherein the packaging step comprises including a plurality of magnetic anastomotic components as part of a kit.

25. The process of claim 24 wherein the packaging step further comprises including at least one delivery device in the kit.

26. The process of claim 20 further comprising microblasting or acid-etching an exterior surface of the component to remove unwanted material, and then coating the compatible with a layer of biocompatible material prior to the packaging step.

27. A process for producing a magnetic anastomotic component suitable for implantation in a patient's body, the process comprising steps of:
providing an anastomotic component having an ability to produce a magnetic field, the component having an exterior surface;
placing a layer of material on a first portion of the exterior surface of the component so as to leave a second portion of the exterior surface of the component uncovered by the material; and
magnetizing the component.

28. The process of claim 27 wherein the material placed on the first portion of the exterior is paramagnetic.

29. The process of claim 28 wherein the second portion of the exterior surface of the component defines an area of concentrated magnetic flux.

30. The process of claim 29 further comprising placing a layer of different material over the exterior surface of the component.

31. The process of claim 30 wherein the different material has diamagnetic properties.

32. The process of claim 29 wherein the second portion of the component defines a continuous area of concentrated flux.

33. A process for producing a magnetic anastomotic component suitable for implantation in a patient's body, the process comprising steps of:
forming an anastomotic component having a desired configuration from a material capable of producing a magnetic field, the component having an exterior surface;
subjecting the component to an acid etching process to remove surface irregularities; and
providing the exterior surface of the component with a layer of biocompatible material.

34. The process of claim 33 wherein the subjecting step is performed by placing the component in a solution containing phosphoric acid.

35. The process of claim 34 wherein the component is placed in the phosphoric acid solution for an amount of time within the range of from about 5 minutes to about 15 minutes.

36. The process of claim 34 further comprising subjecting the solution to electric potential after the acid etching step

37. The process of claim 33 further comprising providing at least a portion of the exterior surface of the component with traction structure for enhancing engagement between the component and the tissue of a vessel.

38. The process of claim 37 wherein the traction structure comprises a surface of the component provided with adhesive.

39. The process of claim 37 wherein the traction structure comprises a surface of the component provided with tissue-gripping elements configured to grip the tissue of a vessel.

40. The process of claim 37 wherein the traction structure comprises a surface of the component provided with a tacky coating configured to stick to vessel tissue.
